(19) United States
(12) Patent Application Publication
(10) Pub. No.: US 2024/0099940 A1
(43) Pub. Date: Mar. 28, 2024

(54) Pacifier and Method to Determine Related Information

(71) Applicant: FB Group ApS, Hinnerup (DK)

(72) Inventor: Henrik Tøth Jensen, Søften Hinnerup (DK)

(21) Appl. No.: 18/520,982

(22) Filed: Nov. 28, 2023

Related U.S. Application Data
(63) Continuation of application No. PCT/DK2022/050118, filed on Jun. 3, 2022.

(30) Foreign Application Priority Data
Jun. 4, 2021 (DK) PA 2021 00602

(51) Int. Cl. A61J 17/00; H04B 5/00

(52) U.S. Cl. CPC A61J 17/101; A61J 17/001; H04B 5/0025; A61J 2205/60

(57) ABSTRACT

A pacifier comprising a base portion, a body portion and a nipple attached to the body portion is disclosed. The pacifier comprises a Near Field Communication (NFC) tag arranged in such a position that when the NFC tag is triggered by the electromagnetic interrogation pulse from a nearby NFC device integrated in the NFC device, the NFC tag transmits digital data in the form of a unique ID, back to the NFC device.

### CROSS-REFERENCE TO RELATED APPLICATIONS

This application is a continuation under 35 U.S.C. 111 of International Patent Application No. PCT/DK2022/050118, filed Jun. 3, 2022, which claims the benefit of and priority to Danish Application No. PA 2021 00602, filed Jun. 4, 2021, each of which is hereby incorporated by reference in its entirety.

### FIELD OF INVENTION

The present invention relates to a pacifier containing an electronic identification device such as a Near Field Communication (NFC) tag or similar passive device.

### BACKGROUND

It is well-known that parents are extremely concerned about the well-being of their babies and this also concerns the use of pacifiers. Accordingly, several studies have been conducted in order to verify the content of chemicals in pacifiers and their potential harmful effect on babies.

Furthermore, it is very important that the pacifier has been subjected to a number of mechanical stress tests to ensure that the pacifier does not break, thereby introducing the risk of fatal consequences. The pacifier's production date is of great importance, as the pacifier's mechanical strength and thus resistance against rupture decreases with the pacifier's production age. Accordingly, the associated risk of fatal consequences in the form of suffocation depends on and increases with the pacifier's production age. Research has also shown that the ageing process can cause creation of chemical compounds in the pacifier, where these chemical compounds were not measurable at the time of production.

Therefore, it is very important to be able to verify the pacifier's production age in a fast and easy manner. It is an object to provide a way to be able to verify the pacifier's production age in a fast and easy manner.

Occasionally it is detected that a released product needs to be recalled. A product recall is typically defined as a request to return to the maker, a batch or an entire production run of a product. Normally, a recall is initiated due to safety concerns or design defects or labelling errors.

US20160367188A1 discloses a pacifier that comprises an implantable oral sensor receptacle. The pacifier is designed to enable parents and care givers many additional metabolic measurements. The pacifier comprises one or more sensors. The pacifier comprises a wireless transceiver chip which is built into the pacifier device enabling the device the ability to send and receive data, commands and other information. In practice, however, it has been found that communication based on an NFC tag requires a specific design in order for the NFC tag to be triggered by the electromagnetic pulse from a nearby NFC device integrated in a mobile device. The NFC tag will only generate and transmit a radio wave signal containing an ID of the pacifier back to the mobile device if specific requirements are met.

Accordingly, it is an object to provide a pacifier that comprises an NFC tag that is configured to be powered by a mobile device, wherein the NFC tag is configured to generate and transmit a radio wave signal containing an ID of the pacifier back to the mobile device.

In the event of a product recall, one typically releases announcements on the respective government agency's website. Moreover, notices can be given in the metropolitan daily newspapers and be advised in news television reports. During such recall, the consumer will be requested to return goods, irrespective of condition, to the selling point for a full refund or modification.

In practice, this recall procedure is very difficult, expensive, time consuming and rather inefficient. Accordingly, it would be desirable to provide an alternative. Accordingly, it is an object to provide an improved and more efficient way of carrying out a recall for pacifiers.

### BRIEF DESCRIPTION

A pacifier according to the present disclosure is a pacifier comprising:
a base portion;
a body portion; and
a nipple attached to the body portion,
   wherein the pacifier comprises an NFC tag configured and arranged in such a position that when the NFC tag is triggered by the electromagnetic pulse from a nearby NFC device integrated in a mobile device, the NFC tag generates and transmits a radio wave signal containing an ID of the pacifier back to the mobile device, wherein:
the pacifier comprises an end portion attached to or integrated into the body portion, wherein the NFC tag is:
a) integrated in the body portion; or
b) arranged at or attached to an end face of the body portion that is covered by the end portion; or
the pacifier comprises an end portion, wherein the NFC tag is placed on, attached to or integrated into the end portion.

Hereby, it is possible to verify the pacifier's production age and other relevant information in a fast and easy manner.

The base portion, body portion and nipple may have any suitable geometry and size. In an embodiment, the base portion and body portion are of similar size and design as base portions, body portions and nipples of standard pacifiers.

The NFC tag is configured and arranged in such a position that when the NFC tag is triggered by the electromagnetic pulse from a nearby NFC device integrated in a mobile device, the NFC tag generates and transmits a radio wave signal containing an ID of the pacifier back to the mobile device. This may be accomplished in several ways.

The NFC device can be considered as the mobile device itself. The NFC device can, however, also be considered as a separate device integrated in the mobile device. In the following, it has to be understood that the mobile device either is an NFC device or comprises an NFC device, wherein the NFC device is capable of generating a changing magnetic field that can induce an electromotive force in a nearby coil of an NFC tag.

In an embodiment, the pacifier comprises an end portion attached to the body portion.

In an embodiment, the NFC tag is integrated in the body portion.

In an embodiment, the NFC tag is arranged at a portion of the body portion that is covered by the end portion.

In an embodiment, the NFC tag is arranged at a portion of an end face of the body portion that is covered by the end portion.

In an embodiment, the NFC tag is attached to a portion of an end face of the body portion that is covered by the end portion.

In an embodiment, the pacifier comprises an end portion, wherein the NFC tag is placed on the end portion.

In an embodiment, the pacifier comprises an end portion, wherein the NFC tag is attached to the end portion.

In an embodiment, the pacifier comprises an end portion, wherein the NFC tag is integrated into the end portion.

In an embodiment, the ID is a unique ID of the pacifier.

Hereby, the pacifier can be identified on the basis of its unique ID (or serial number). This ID can be used to identify the individual pacifier or the production batch it originates from. Accordingly, it is possible to verify the pacifier's production age and other relevant information in a fast and easy manner.

The NFC tag comprises a coil shaped to form a radio receiver (antenna). When a changing magnetic field is applied to the coil, an electric current will flow in the coil. Hereby, the NFC tag, by using its integrated energy harvester (the coil) produces enough energy to generate and transmit a radio wave signal containing information about the unique ID of the pacifier. When an NFC device integrated in an NFC device such as a mobile phone or a tablet is placed in close proximity to the NFC tag, the NFC tag will harvest sufficient energy to generate and transmit the radio wave signal, which will be detected by the NFC device.

In an embodiment, the pacifier comprises an end portion attached to or integrated into the body portion, wherein the NFC tag is attached to an end face of the body portion that is covered by the end portion. Hereby, it is possible to seal the NFC tag in an efficient manner that at the same time allows the NFC tag to be brought into close proximity to a mobile device such as a mobile phone, a personal computer (PC) or a tablet.

In an embodiment, the pacifier comprises an end portion and the NFC tag is attached to or integrated into the end portion. This embodiment allows the NFC tag to be brought into close proximity to a mobile device such as a mobile phone or a tablet.

In an embodiment, the NFC tag is formed as a label that is attached to a structure of the pacifier. Hereby, it is possible to attach the NFC tag in a simple and easy manner. Moreover, it is an advantage that the height of the NFC tag is small. The label may comprise an adhesive layer configured to be brought into contact with the structure of the pacifier in order to attach the label to the surface.

In an embodiment, the NFC tag is formed as a label that is attached to a surface of the body portion.

In an embodiment, the NFC tag is formed as a label that is attached to a structure of the body portion.

In an embodiment, the NFC tag is formed as a label that is attached to a surface of the end portion.

In an embodiment, the NFC tag is formed as a label that is attached to a structure of the end portion.

In an embodiment, the NFC tag is arranged in a cavity provided in the pacifier. Hereby, it is possible to avoid that the NFC tag protrudes from the surface of the remaining surface of the pacifier.

In an embodiment, the NFC tag is arranged in a cavity provided in the body portion.

In an embodiment, the NFC tag is arranged in a cavity provided between the body portion and the end portion.

In an embodiment, the NFC tag is arranged in a cavity provided in the end portion.

It may be an advantage that the cavity is sealed or covered by a seal or cover element. Hereby, it is possible to protect the NFC tag against water, moisture, and impact from the surroundings.

In an embodiment, the NFC tag is integrated in the pacifier during an injection molding process. Hereby, it is possible to integrate the NFC tag in a manner that leaves no traces from the attachment of the NFC tag. It is possible to manufacture a pacifier that has the same design as pacifiers without an integrated NFC tag.

In an embodiment, the NFC tag comprises a coil, wherein the diameter of the coil of the NFC tag is in the range 0.5-5.0 cm.

In an embodiment, the diameter of the coil of the NFC tag is in the range 0.9-1.5 cm. In an embodiment, the diameter of the coil of the NFC tag is in the range 1.0-1.4 cm. In an embodiment, the diameter of the coil of the NFC tag is in the range 1.1-1.3 cm. In an embodiment, the diameter of the coil of the NFC tag is in the range 1.0-2.0 cm. In an embodiment, the diameter of the coil of the NFC tag is in the range 2.0-3.0 cm. In an embodiment, the diameter of the coil of the NFC tag is in the range 3.0-4.0 cm. In an embodiment, the diameter of the coil of the NFC tag is in the range 4.0-5.0 cm.

In an embodiment, the pacifier comprises a handle rotatably attached to the body portion.

In an embodiment, the pacifier comprises an end portion, wherein the NFC tag is arranged between the body portion and the end portion, wherein the gap between the body portion and the end portion is sealed.

In an embodiment, the gap between the body portion and the end portion is sealed by a seal made by ultrasonic welding.

In an embodiment, the number of turns of the coil is at least two.

In an embodiment, the number of turns of the coil is at least three.

In an embodiment, the number of turns of the coil is at least four.

In an embodiment, the number of turns of the coil is at least five.

It may be an advantage that the one or more turns of the coil are arranged in a single plane. Hereby the coil takes up less space and a more compact NFC tag can be provided.

In an embodiment, the end portion comprises a planar outer surface, wherein the distance between the NFC tag and the outer surface is less than 3 mm.

In an embodiment, the distance between the NFC tag and the outer surface is less than 2 mm.

In an embodiment, the distance between the NFC tag and the outer surface is less than 1 mm.

By providing a short distance between the NFC tag and the outer surface, it becomes easier to energize the NFC tag by using a mobile device.

A system according to the present disclosure is a system comprising:
a pacifier;
a mobile device comprising an NFC device configured to detect a radio wave signal containing the ID of the pacifier.
a web-based service accessible via the Internet,
   wherein the web-based service is configured to:
receive data from mobile device, wherein the data contains the unique ID of the pacifier;
send data related to the unique ID of the pacifier to the mobile device.

Hereby, the user of the mobile device receives the requested information.

The web-based service may be a server accessible via the Internet.

In an embodiment, the mobile device has an installed program that allows the user of the mobile device to select from a list of predefined information, wherein the list includes information related to the pacifier (e.g. the production date of the pacifier). In principle, the list may contain any relevant information.

In an embodiment, the mobile device has an installed program that allows the user of the mobile device to select from a list of predefined information, wherein the list includes information containing the chemical contents of the materials that the pacifier is made from. Hereby, it is possible to access adequate information about the chemical contents of the materials that the pacifier is made from.

In an embodiment, the mobile device has an installed program that allows the user of the mobile device to select from a list of predefined information, wherein the list includes information containing the location of the production site of the pacifier. Hereby, it is possible to access adequate information about the location of the production site of the pacifier.

In an embodiment, the mobile device has an installed program that allows the user of the mobile device to select from a list of predefined information, wherein the list includes information containing the energy consumption relevant for the production and/or transportation of the pacifier. Hereby, it is possible to access adequate information about the energy consumption relevant for the production and/or transportation of the pacifier.

In an embodiment, the mobile device has an installed program that allows the user of the mobile device to select from a list of predefined information, wherein the list includes information containing the carbon dioxide emissions relevant for the production and/or transportation of the pacifier. Hereby, it is possible to access adequate information about the production and/or transportation of the pacifier.

A system according to the present disclosure makes it possible to verify the pacifier's production age in a fast and easy manner.

The requested information may be the location of the production site of the pacifier. The requested information may be the energy consumption relevant for the production and/or transportation of the pacifier.

The requested information may be the carbon dioxide emissions relevant for the production and/or transportation of the pacifier.

Hereby, the user of the mobile device can request any type of information from the list in a fast and easy manner.

A method according to the present disclosure is a method for providing a unique ID of a pacifier that comprises:
a base portion;
a body portion; and
a nipple attached to the body portion,
   wherein the method comprises the steps of:
providing a pacifier that comprises an NFC tag arranged in such a position that when the NFC tag is triggered by the electromagnetic pulse from a nearby NFC reader device integrated in a mobile device, the NFC tag generates and transmits a radio wave signal containing an ID of the pacifier back to the mobile device;
bringing the NFC device of the mobile device into a sufficiently small distance from the NFC tag so that the NFC tag generates and transmits a radio wave signal containing the ID of the pacifier;
receiving the ID of the pacifier by the mobile device,
   wherein:
the pacifier comprises an end portion attached to or integrated into the body portion, wherein the NFC tag is:
a) integrated in the body portion; or
b) arranged at or attached to an end face of the body portion that is covered by the end portion; or
the pacifier comprises an end portion, wherein the NFC tag is placed on, attached to or integrated into the end portion.

Hereby, the method makes it possible to detect the ID of the pacifier by the mobile device in an easy and user-friendly manner.

In an embodiment, the method comprises the step of:
sending the ID of the pacifier to a web-based service that is configured to receive data from mobile device, wherein the data contains the unique ID of the pacifier;
ensuring that the web-based service sends data related to the unique ID of the pacifier to the mobile device.

Hereby, it is possible to access data related to the unique ID of the pacifier and make these data available on the mobile device.

In an embodiment, the method comprises the step of running a program installed on the mobile device, wherein the installed program is configured to allow the user of the mobile device to select from a list of predefined information, wherein the list includes information related to the pacifier (e.g. the production date of the pacifier). Hereby, it is possible to provide information containing the production date of the pacifier in a fast and easy manner.

In an embodiment, the method comprises the step of running a program installed on the mobile device, wherein the installed program is configured to allow the user of the mobile device to select from a list of predefined information, wherein the list includes information containing the chemical contents of the materials that the pacifier is made from. Hereby, it is possible to provide information containing the chemical contents of the materials that the pacifier is made from in a fast and easy manner.

In an embodiment, the method comprises the step of running a program installed on the mobile device, wherein the installed program is configured to allow the user of the mobile device to select from a list of predefined information, wherein the list includes information containing the location of the production site of the pacifier. Hereby, it is possible to provide information containing the location of the production site of the pacifier in a fast and easy manner.

In an embodiment, the method comprises the step of running a program installed on the mobile device, wherein the installed program is configured to allow the user of the mobile device to select from a list of predefined information, wherein the list includes information containing the energy consumption relevant for the production and/or transportation of the pacifier. Hereby, it is possible to provide information containing the energy consumption relevant for the production and/or transportation of the pacifier in a fast and easy manner.

In an embodiment, the method comprises the step of running a program installed on the mobile device, wherein the installed program is configured to allow the user of the mobile device to select from a list of predefined information, wherein the list includes information containing the carbon dioxide emissions relevant for the production and/or transportation of the pacifier. Hereby, it is possible to provide information containing the carbon dioxide emissions relevant for the production and/or transportation of the pacifier in a fast and easy manner.

A method for recalling a number of pacifiers according to the present disclosure is a method that comprises the following steps:
providing a number of pacifiers according to the present disclosure;
for each pacifier, bringing the NFC device of the mobile device into a sufficiently small distance from the NFC tag so that the NFC tag generates a radio wave signal containing the ID of the pacifier;
for each pacifier, receiving the ID of the pacifier by the corresponding mobile device;
for each pacifier, sending the ID of the pacifier to a web-based service that is configured to receive data from the respective mobile device, wherein the data contains the unique ID of the pacifier;
receiving the ID of the pacifiers by the web-based service;
sending recall information to each of the mobile devices by the web-based service.

Hereby, it is possible to provide an alternative to the prior art recall procedures. By detecting the identity of the mobile devices that send the ID of the pacifiers to a web-based service it is possible to inform the user of the pacifier that it needs to be recalled. Hereby, it is possible to provide an improved and more efficient way of carrying out a recall for pacifiers.

The “recall information” sent to the mobile devices by the web-based service may include any suitable information such as the location (address) where the pacifiers can be returned.

On the basis of the unique ID of each of the pacifiers, it is possible to ensure that the web-based service only sends “recall information” to the relevant users.

An object of the disclosure can be achieved by a method for manufacturing a pacifier, wherein the method comprises the following steps:
arranging the NFC tag between the body portion and the end portion of a pacifier; and
joining the body portion and the end portion by a sealing procedure.

In an embodiment, the sealing procedure is ultrasonic welding.

Hereby, it is possible to provide a waterproof seal. Thus, the pacifier can be submerged into a liquid without being damaged.

### BRIEF DESCRIPTION OF THE DRAWINGS

The systems and methods will become more fully understood from the detailed description given herein below. The accompanying drawings are given by way of illustration only, and thus, they are not limitative of the present disclosure. In the accompanying drawings:

FIG. 1 shows a perspective end view of a pacifier according to an embodiment;

FIG. 2 shows another perspective end view of a pacifier according to an embodiment;

FIG. 3 shows a perspective side view of a pacifier according to an embodiment;

FIG. 4 shows a base portion of a pacifier according to an embodiment together with a first plane extending along the base portion and a second orthogonal plane;

FIG. 5 shows a mobile phone arranged above a pacifier according to an embodiment;

FIG. 6 shows the mobile phone shown in FIG. 5 arranged at a shorter distance above the pacifier;

FIG. 7 shows a perspective view of a body portion and a corresponding end portion of a pacifier according to an embodiment;

FIG. 8A shows a perspective view of the body portion and a corresponding end portion shown in FIG. 7;

FIG. 8B shows several components of a pacifier according to an embodiment in order to illustrate one way to attach the nipple to the body portion of the pacifier;

FIG. 9A shows a perspective view of a base portion of a pacifier according to an embodiment;

FIG. 9B shows a perspective view of another base portion of a pacifier according to an embodiment;

FIG. 9C shows a perspective view of a further base portion of a pacifier according to an embodiment;

FIG. 9D shows a perspective view of yet another base portion of a pacifier according to an embodiment;

FIG. 9E shows a perspective view of a further body portion of a pacifier according to an embodiment;

FIG. 9F shows a perspective view of yet another body portion of a pacifier according to an embodiment;

FIG. 10 shows the communications path of a method according to an embodiment;

FIG. 11 shows a flowchart illustrating the process steps of a method according to an embodiment;

FIG. 12A shows a flowchart illustrating the process steps of another method according to an embodiment;

FIG. 12B shows a schematic, cross-sectional view of a portion of a pacifier according to an embodiment; and

FIG. 12C shows the portion of the pacifier shown in FIG. 12B.

### DETAILED DESCRIPTION

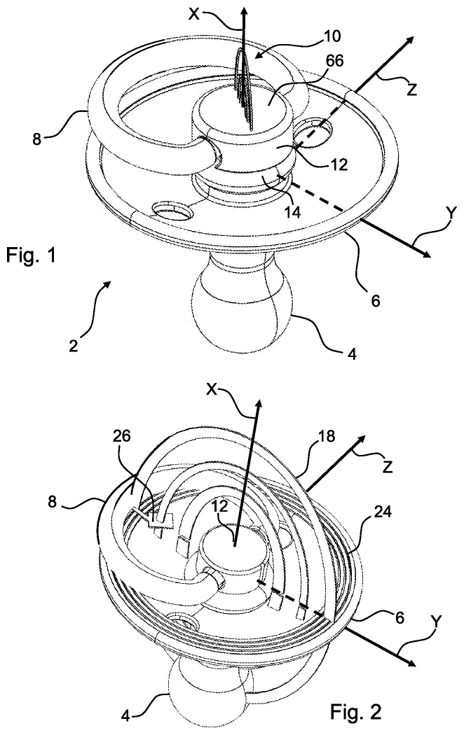

Referring now in detail to the drawings for the purpose of illustrating embodiments of the present invention, a pacifier 2 of the present disclosure is illustrated in FIG. 1.

FIG. 1 illustrates a perspective end view of a pacifier 2 according to an embodiment. The pacifier 2 comprises a body portion 14 and a nipple 4 attached to the body portion 14. An end portion 12 is attached to and hereby closes the distal end of the body portion 14. The end portion 12 may be fixed to the body portion 14 by ultrasonic welding. The end portion 12 has a planar outer surface 66.

The pacifier 2 comprises a handle 8 rotatably attached to the body portion 14. A passive Near Field Communication (NFC) tag that comprises no internal power source is configured to be powered by the electromagnetic energy transmitted from an NFC device integrated in an NFC device such as a mobile phone or a tablet. The NFC device is configured to communicate using a set of communication protocols for communication between two electronic devices over a short distance (typically a distance of 6 cm or less).

When the NFC device such as a mobile phone (as shown in FIG. 6) is arranged at a close distance from the NFC tag, the magnetic field of the NFC device induces a current in a coil of the NFC tag. Hereby, the NFC tag is activated and powered by the harvested energy. The NFC tag is configured to send out a wireless signal containing a unique identification (ID) upon being activated. The NFC tag comprises a radio transmitter configured to transmit a radio wave signal containing the unique ID or information that makes it possible to verify the unique ID. When triggered by the electromagnetic interrogation pulse from the nearby NFC reader device integrated in the NFC device, the tag transmits the radio wave signal back to the NFC device. In an embodiment, the unique ID is an identifying inventory number.

The radio wave signal 10 generated by the NFC tag is indicated in FIG. 1. A range the radio wave signal 10 is transmitted from the end portion of the pacifier 2 can be seen. Since the magnetic field provided by mobile devices, such as mobile phones, has limited intensity/strength, the position of the NFC tag must be selected in such a manner that the distance between the NFC tag and the NFC device is sufficiently small to ensure that the magnetic field of the NFC device induces a current in a coil of the NFC tag so that the NFC tag is activated and powered by the harvested energy. In practice, it is difficult to achieve. The embodiments herein, however, provide suitable solutions to this problem.

The nipple 4 extends along the longitudinal axis X of the pacifier 2. The pacifier 2 comprises a base portion 6 designed as a shield that limits the insertion depth of the nipple 4. The base portion 6 basically extends along a plane extending perpendicular to the longitudinal axis X. This plane is spanned by the lateral axes Y, Z. The base portion 6, however, is not planar, but forms a concave geometry (seen from a position along the positive part of the X axis).

When the NFC tag has to be integrated into the pacifier 2, it is essential to place the NFC tag in a position that makes it possible to energise the NFC tag by a nearby NFC device. Due to the anatomy of the pacifier 2 and the fact that the optimal reading angle of the NFC tag is perpendicular to the plane, along which the NFC tag extends, a desirable location will be in the distal end of the centrally arranged body portion 14. From this position it is possible to bring a mobile phone into close proximity to the body portion 14 and thus the NFC tag. In this manner, the distance between the NFC tag and the NFC device can be small enough to allow the NFC device to energize the NFC tag successfully to enable activation of the NFC tag (so that the NFC tag generates and transmits a radio wave signal 10 containing the ID of the pacifier 2.

In an embodiment, the NFC tag is mounted directly on the end face of the body portion 14. In an embodiment, the NFC tag is mounted on the body portion 14 as a label. In an embodiment, the label is covered by a sealing layer. This embodiment is useful in relation to “reading distance”, however, the NFC tag may be exposed to wear and tear e.g. when boiling the pacifier 2.

FIG. 2 illustrates another perspective end view of a pacifier 2 according to an embodiment. The pacifier 2 shown in FIG. 2 comprises a body portion 14, a nipple 4 attached to the body portion 14 and an end portion 12 attached to and hereby closing the distal end of the body portion 14 (like the pacifier 2 shown in FIG. 1). Besides, the pacifier 2 comprises a handle 8 rotatably attached to the body portion 14.

The pacifier 2 comprises an NFC tag 26 placed at the base portion 6. The NFC tag 26 comprises a coil 24 constituting an antenna which may also be considered to be a receiver. When a changing magnetic field is applied to the coil 24, an electric current will flow in the coil. Hereby, the NFC tag 26, by using its integrated energy harvester (the coil 24), produces a sufficient amount of energy to generate and transmit a radio wave signal 18 that contains information about the unique ID of the pacifier 2. When an NFC mobile phone or a tablet is placed in close proximity to the NFC tag 26, the NFC tag 26 will harvest sufficient energy to generate and transmit a radio wave signal 18, which will be detected by the NFC device due to the short distance.

It should be noted that the NFC tag 26 may be arranged in another position than the one shown in FIG. 2.

In an embodiment, the NFC tag 26 is embedded in the base portion 6 (e.g. during an injection molding process).

In an embodiment, the NFC tag 26 is placed in a cavity (not shown) provided in the base portion 6. This cavity may be covered with a sealing material when the NFC tag 26 has been placed in a cavity.

Moreover, it should be emphasized that the illustration of the radio wave signal 18 is merely illustrative. In an embodiment, the coil 24 comprises a number of turns arranged in and extending along a plane perpendicular to the longitudinal axis X of the pacifier 2. This means that the turns of the coil 24 are arranged in and extend along a plane parallel to the plane spanned by the Y axis and the Z axis.

In another embodiment, the NFC tag 26 and its coil 24 are designed as a label mounted on the front or back of base portion 6. By arranging the NFC tag 26 and its coil 24 on the base portion 6, it is possible to apply a coil 24 having a large diameter (compared to the diameter of the base portion 6). The large diameter of coil 24 of the NFC tag 26 makes it possible to provide a significantly greater reading distance. Accordingly, the NFC tag 26 can be read both on the nipple side and on the rear side of the pacifier 2 where the handle 8 is located. In an embodiment, the turns of the coil 24 are arranged in a circular geometry. In another embodiment, the turns of the coil 24 are arranged to form a polygon. In an embodiment, the turns of the coil 24 are arranged to form a rectangle. In an embodiment, the turns of the coil 24 are arranged to form a pentagon. In an embodiment, the turns of the coil 24 are arranged to form a hexagon. In an embodiment, the turns of the coil 24 are arranged to form an octagon. In an embodiment, the turns of the coil 24 are arranged to form a decagon.

The effect of increasing the size (diameter) of the coil 24 is, however, limited in practice because many NFC devices such as mobile phones only generate a magnetic field having a limited magnetic field intensity. Since larger NFC tag coils 24 generally require a larger amount of energy to create power to the NFC tag chip, an enlargement of the coil 24 cannot compensate for the limited magnetic field intensity of the NFC devices. Accordingly, the NFC device (e.g. an NFC mobile phone) simply needs to be held closer to the coil 24 of the NFC tag 20.

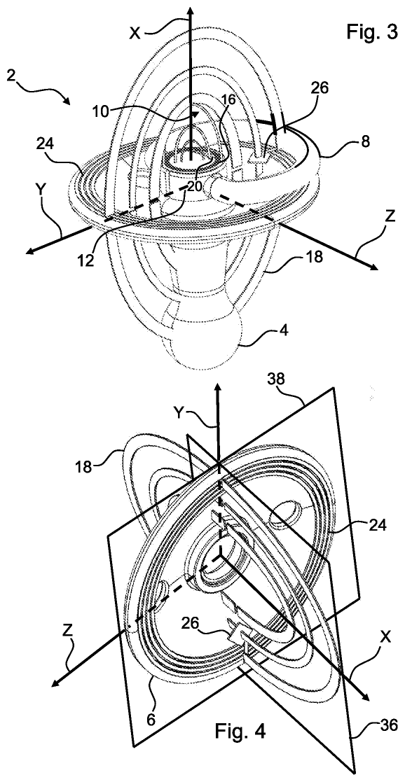

FIG. 3 illustrates a perspective side view of a pacifier 2 according to an embodiment. The pacifier 2 comprises some of the same components as the pacifier 2 shown in FIG. 1, namely: a body portion 14, a nipple 4 attached to the body portion 14, an end portion 12 attached to the distal end of the body portion 14 and a handle 8 rotatably attached to the body portion 14.

The pacifier 2, however, comprises both a first passive NFC tag 20 and a second passive NFC tag 26. The first passive NFC tag 20 corresponds to the one referred to with reference to FIG. 1. The second passive NFC tag 26, however, corresponds to the one shown in and explained with reference to FIG. 2. Therefore, the pacifier 2 is configured to generate two radio wave signals when an NFC device is brought into close proximity to the coils 16, 24 of the first passive NFC tag 20 and the second passive NFC tag 26, respectively. The first passive NFC tag 10 is attached to the end surface of the end portion 12. This may be accomplished using a glue (e.g. using an NFC tag 20 shaped as a label). The NFC tag 20 may alternatively be placed on the end portion 12 during or after injection molding the end portion 12. In an embodiment, the NFC tag 20 is placed in a small cavity provided in the end portion 12, whereafter a cover member is attached to the NFC tag 20 in order to seal the NFC tag 20 and secure it in a sufficient manner to the end portion 12.

In an embodiment, the pacifier 2 is configured to generate a first radio wave signal when an NFC device is brought into close proximity to one of the coils 16, 24 and a second radio wave signal when an NFC device is brought into close proximity to one of the coils 16, 24. In an embodiment, the first radio wave signal and the second radio wave signal differ from one another.

In an embodiment, the first radio wave signal and the second radio wave signal are intended to transmit data containing the same unique ID. Hereby, it is possible to minimize the risk of malfunction (since both NFC tags 20, 26 generate the same unique ID).

FIG. 4 illustrates a base portion 6 of a pacifier according to an embodiment. FIG. 4 shows a first plane 36 extending along the base portion 6 as well as a second orthogonal plane 38. It can be seen that the base portion 6 comprises a passive NFC tag 26 having a circular shaped coil 24 arranged near the periphery of the base portion 6.

The first plane 36 is spanned by the longitudinal axis X and the lateral axis Y of the pacifier. The NFC tag 26 comprises a chip shaped as a small planar element. The chip of the NFC tag 26 may be attached to the base portion 6 or be integrated therein using any suitable means including gluing, injection molding or mechanical attachment structures.

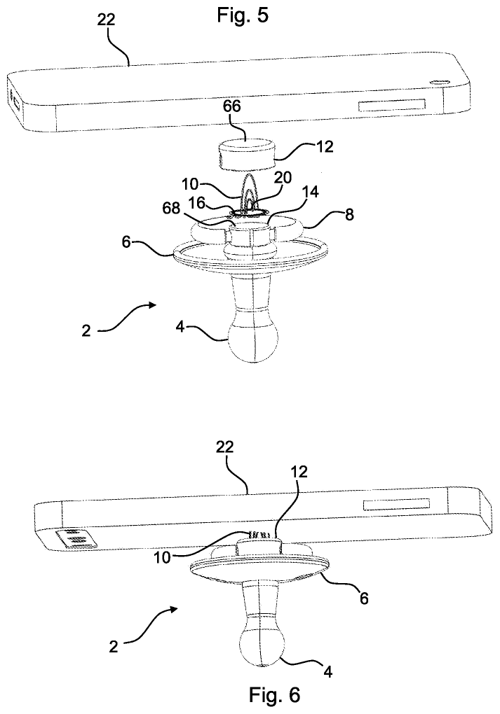

FIG. 5 illustrates a mobile phone 22 arranged above a pacifier 2 according to an embodiment. The pacifier 2 corresponds to the one shown in and explained with reference to FIG. 1. In FIG. 5, however, the NFC tag 20 and its coil 16 has been removed from the body portion 14 for illustrative purposes. Likewise, the end portion 12 has been removed from the body portion 14 to make the NFC tag 20 and its coil 16 visible.

When the mobile phone 22 is arranged in the position as illustrated in FIG. 5, the range (intensity) of the magnetic field generated by the mobile phone 22 is not large enough to enable the NCF tag of the pacifier 2 to generate and send a radio wave signal 10 even though the mobile phone 22 is configured to detect the unique ID contained in radio wave signal 10. The end portion 12 has a planar outer surface 66. The body portion 14 has a planar distal surface 68.

FIG. 6, however, illustrates the mobile phone 22 shown in FIG. 5 arranged at a shorter distance above the pacifier 2. When the mobile phone 22, however, is arranged in a position corresponding to the one shown in FIG. 6, the range (intensity) of the magnetic field generated by the mobile phone 22 is large enough to allow the NFC tag to harvest sufficient energy to generate and transmit a radio wave signal 10 to the mobile phone 22. Accordingly, the mobile phone 22 detects the unique ID contained in the radio wave signal 10.

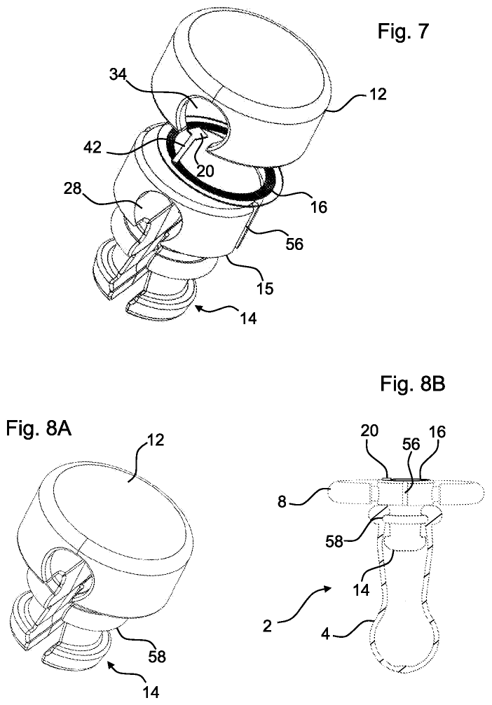

FIG. 7 illustrates a perspective view of a body portion 14 and a corresponding end portion 12 of a pacifier according to an embodiment. This embodiment corresponds to the one shown in FIG. 5. For illustrative purposes, the end portion 12 has been removed from the body portion 14. Likewise, the NFC tag 20 and its coil 16 have been removed from the body portion 14 to make these components visible. It can be seen that the NFC tag 20 comprises a connection portion 42 extending between the chip portion of the NFC tag 20 and the coil 16 of the NFC tag 20. The connection portion 42 is electrically conducting and may be shaped as a wire or an electrically conducting bond.

It can also be seen that the end portion 12 is provided with holes 34 for receiving a handle like the one shown in FIG. 5. The body portion 14 comprises a distal portion 15 provided with a through bore 28. The through bore 28 is also arranged and configured to receive a handle like the one shown in FIG. 5.

The distal portion 15 comprises two track structures 56 configured to engage with corresponding guide structures (not shown) provided at the inside surface of the end portion 12 in order to position the end portion 12 in the correct position relative to the distal portion 15 of the body portion 14. Hereby, it can easily be ensured that the holes 34 of the distal portion 15 of the body portion 14 are aligned with the through bore 28 of the end portion 12 to allow for insertion of a handle like the one shown in FIG. 5.

FIG. 8A illustrates a perspective view of the body portion 14 and the corresponding end portion 12 shown in FIG. 7. It can be seen that the end portion 12 has been attached to the portion of the body portion 14. Moreover, it can be seen that the through bore of the end portion 12 is aligned with the holes of the distal portion of the body portion 14. Accordingly, a handle like the one shown in FIG. 5 can be inserted into the channel defined by the through bore of the end portion 12 and the holes of the distal portion of the body portion 14. The body portion 14 comprises a protruding structure 58 arranged and configured for attachment of a nipple (see FIG. 8B).

FIG. 8B illustrates a pacifier 2 according to an embodiment. One can see a cross-sectional view of a nipple 4 surrounding a protruding structure 58 of a body portion 14. A handle 8 is rotatably mounted to the body portion 14. An NFC tag 20 having a coil 16 is provided on the end portion of the body portion 14. For illustrative purposes, the base portion has been removed from the pacifier 2. The body portion 14 comprises one or more track structures 56 configured to engage with corresponding guide structures provided at the inside surface of an end portion, such as the one shown in FIG. 8A.

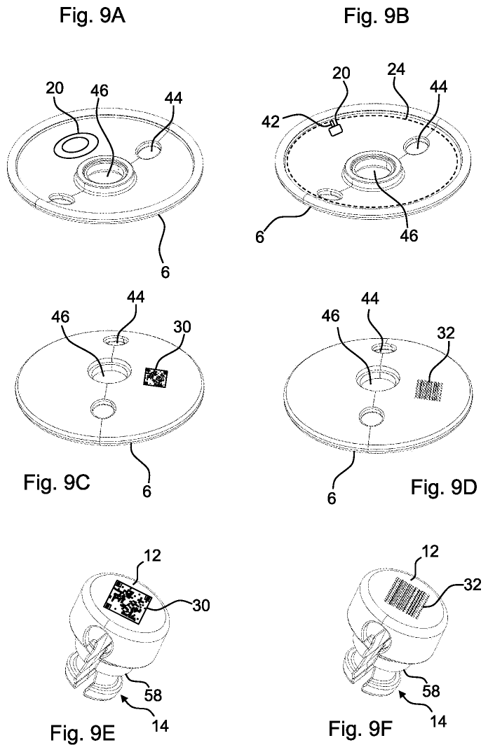

FIG. 9A illustrates a perspective view of a base portion 6 of a pacifier according to an embodiment. The base portion 6 comprises an NFC tag 20 having a coil provided at the front surface of the base portion 6. The base portion 6 is provided with a centrally arranged opening 46 for receiving the proximal portion of a nipple as the proximal portion of a body portion (such as the one shown in FIG. 8B). The base portion 6 is provided with two ventilation holes 44.

FIG. 9B illustrates a perspective view of another base portion 6 of a pacifier according to an embodiment. The base portion 6 comprises an NFC tag 20 having a connection portion 42 and a coil 24 (indicated by a dotted line) provided at the front surface of the base portion 6. The coil 24 is circular and extends near the periphery of the base portion 6. Hereby, the base portion 6 has a large diameter which facilitates its ability to harvest energy by induction from a nearby NFC device. The connection portion 42 is arranged and configured to electrically connect the coil 24 and the chip portion of the NFC tag 20. The base portion 6 is provided with a centrally arranged opening 46 and two ventilation holes 44 like the base portion 6 shown in FIG. 9A.

FIG. 9C illustrates a perspective view of a further base portion 6 of a pacifier according to an embodiment. The geometry of the base portion 6 corresponds to the one shown in FIG. 9A and in FIG. 9B. The base portion 6 comprises a coded ID 30 designed as a two-dimensional barcode such as a Quick Response (QR) code containing a unique identifier (unique ID) of the pacifier having the base portion 6.

FIG. 9D illustrates a perspective view of yet another base portion 6 of a pacifier according to an embodiment. The geometry of the base portion 6 corresponds to the one shown in FIG. 9A, FIG. 9B and FIG. 9C. The base portion 6 comprises a coded ID 32 designed as a barcode that contains a unique identifier (unique ID) of the pacifier having the base portion 6.

FIG. 9E illustrates a perspective view of a body portion 14 of a pacifier according to an embodiment. The geometry of the body portion 14 corresponds to the one shown in FIG. 8A. The body portion 14 comprises a coded ID 30 designed as a two-dimensional barcode such as a Quick Response (QR) code containing a unique identifier (unique ID) of the pacifier having the body portion 14.

FIG. 9F illustrates a perspective view of yet another body portion 14 of a pacifier according to an embodiment. The geometry of the body portion 14 corresponds to the one shown in FIG. 8A. The body portion 14 comprises a coded ID 32 designed as a barcode that contains a unique identifier (unique ID) of the pacifier having the body portion 14.

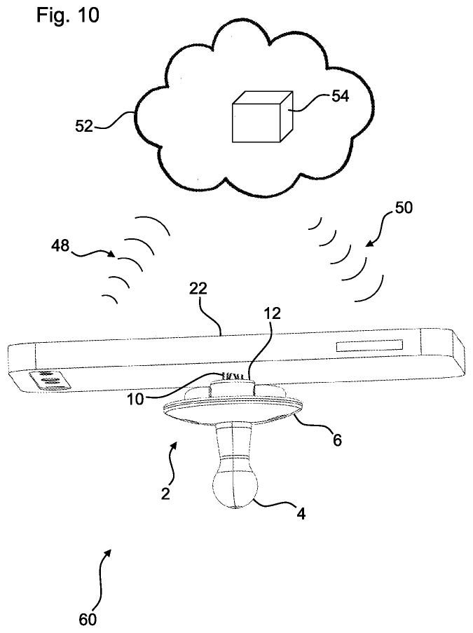

FIG. 10 illustrates the communications path of a method and a system 60 according to an embodiment. The system 60 comprises a pacifier 2 according to an embodiment, a web-based service 54 and a mobile device 22. The mobile device 22 and the pacifier 2 correspond to the ones shown in FIG. 5 and in FIG. 6. The web-based service 54 may be a server connected to and accessible via the Internet 52. The mobile device 22 may be a mobile phone or a tablet by way of example.

By bringing the mobile device 22 into close proximity to the NFC tag of the pacifier 2, the coil of the NFC tag of the pacifier 2 generates and transmits a radio wave signal 10 that is read by the mobile device 22. The radio wave signal 10 reveals the unique ID of the pacifier 2.

The mobile device transmits data 48 to the web-based service 54 via the Internet 52. When the web-based service 54 receives the data 48 that contains the unique ID of the pacifier 2, the web-based service 54 sends data 50 related to the unique ID of the pacifier 2 to the mobile device 22. Hereby, the user of the mobile device 22 receives the requested information.

The requested information may be the production date of the pacifier 2. Hereby, it is possible to verify the pacifier's production age in a fast and easy manner.

The requested information may be the chemical contents of the materials that the pacifier 2 contains. Hereby, it is possible to access adequate information about the chemical contents of the materials that the pacifier 2 is made from.

The requested information may be the location of the production site of the pacifier 2. The requested information may be the energy consumption relevant for the production and/or transportation of the pacifier 2.

The requested information may be the carbon dioxide emissions relevant for the production and/or transportation of the pacifier 2.

The mobile device 22 may use an installed program (application software designed to run on the mobile device 22). In an embodiment, the system 60 comprises a mobile device 22 using an installed program that allows the user of the mobile device 22 to select from a list of predefined information. Hereby, the user of the mobile device 22 can request any type of information from the list in a fast and easy manner.

In case of product recall, the web-based service 54 sends data 50 informing that the pacifier 2 needs to be returned or be discarded to the mobile device 22. Hereby, the user of the mobile device 22 receives the recall information. Accordingly, the user can return or discard the pacifier.

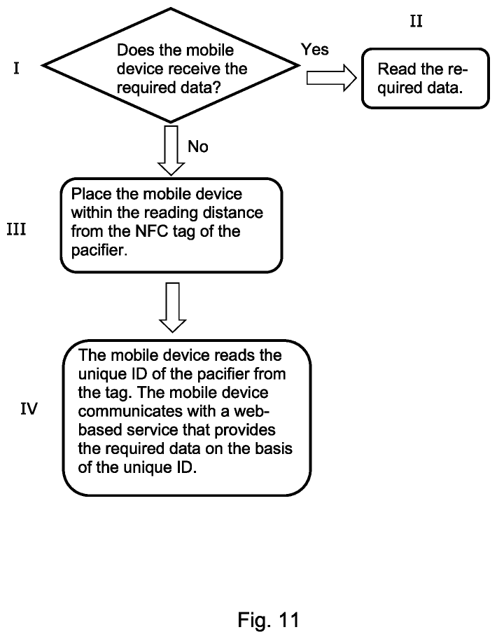

FIG. 11 illustrates a flowchart illustrating the process steps of a method according to an embodiment. In the first step “I” one has to verify if the mobile device receives the required data. If the mobile device receives the required data, the user of the mobile device is satisfied because the requested data are available on the mobile device as shown in second step “II”.

If, on the other hand, the mobile device does not receive the required data, the user of the mobile device must place the mobile device within the reading distance from the NFC tag of the pacifier as illustrated in step “III”.

When the user of the mobile device has placed the mobile device within the reading distance from the NFC tag of the pacifier (as illustrated in step IV), the mobile device communicates with a web-based service that provides the required data on the basis of the unique ID of the pacifier. Now, the user of the mobile device is satisfied because the requested data are available on the mobile device.

It should be noted that step II corresponds to step IV.

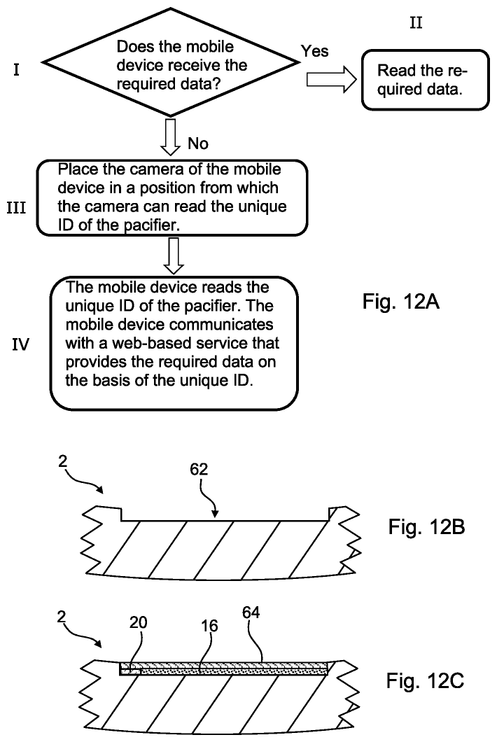

FIG. 12A illustrates a flowchart illustrating the process steps of another method according to an embodiment.

In the first step “I” one has to verify if the mobile device receives the required data. If the mobile device receives the required data, the user of the mobile device is satisfied because the requested data are available on the mobile device as shown in second step “II”.

If, on the other hand, the mobile device does not receive the required data, the user of the mobile device must place the camera of the mobile device in a position from which the camera can read the unique ID of the pacifier as illustrated in step “III”. The unique ID may be one as shown in and explained with reference to FIG. 9C or FIG. 9D.

When the user of the mobile device has placed the mobile device in a position from which the camera can read the unique ID of the pacifier, the mobile device communicates with a web-based service that provides the required data on the basis of the unique ID of the pacifier (as illustrated in step IV). Now, the user of the mobile device is satisfied because the requested data are available on the mobile device.

It should be noted that step II corresponds to step IV.

FIG. 12B illustrates a schematic, cross-sectional view of a portion of a pacifier 2 according to an embodiment provided with a cavity 62. The portion may be a part of the base portion, the body portion or the end portion of the pacifier 2, such as the one shown in FIG. 5.

FIG. 12C illustrates the portion of the pacifier 2 shown in FIG. 12B in a configuration in which an NFC tag 20 having a coil 16 has been placed in the cavity 62. A seal or cover element 64 has been attached on the top side of the NFC tag 20 having a coil 16 in order to sealingly close the cavity. In an embodiment, the seal or cover element 64 is configured to hermetically seal the cavity 62.

### LIST OF REFERENCE NUMERALS

2 Pacifier
4 Nipple
6 Base portion
8 Handle
10 Radio wave signal
12 End member
14 Body portion
15 Distal portion
16 Coil
18 Radio wave signal
20 Tag
22 Mobile phone
24 Coil
26 Tag
28 Through bore
30 Coded ID
32 Coded ID
34 Hole
36 First plane
38 Second plane
40 A
42 Connection portion
44 Hole
46 Opening
48, 50 Data
52 Internet
54 Web-based service
56 Track structure
58 Protruding structure
I, II, III, IV Step
X, Y, Z Axis
60 System
62 Cavity
64 Seal or cover element
66 Outer surface
68 Surface

What is claimed is:

1. A pacifier comprising:
a base portion;
a body portion;
a nipple attached to the body portion;
a Near Field Communication (NFC) tag configured and arranged in a such a position that when the NFC tag is triggered by an electromagnetic pulse from a NFC device integrated in a mobile device, the NFC tag generates a radio wave signal containing an ID of the pacifier back to the mobile device,
an end portion attached to or integrated into the body portion, wherein the NFC tag is:
a) integrated in the body portion;
b) arranged at or attached to an end face of the body portion that is covered by the end portion; or
c) placed on, attached to or integrated into the end portion.

2. The pacifier according to claim 1, wherein the ID is a unique ID of the pacifier.

3. The pacifier according to claim 1, wherein the NFC tag comprises a coil shaped to form a radio receiver.

4. The pacifier according to claim 1, wherein the NFC tag is arranged between the body portion and the end portion, and a gap between the body portion and the end portion is sealed.

5. The pacifier according to claim 4, wherein the gap between the body portion and the end portion is sealed by ultrasonic welding.

6. The pacifier according to claim 1, wherein the NFC tag is formed as a label that is attached to a structure of the pacifier.

7. The pacifier according to claim 1, wherein the NFC tag is arranged in a cavity provided in the pacifier.

8. The pacifier according to claim 7, wherein the cavity is sealed or covered by a seal or cover element.

9. The pacifier according to claim 1, wherein the NFC tag is integrated in the pacifier during an injection molding process.

10. The pacifier according to claim 1, wherein the NFC tag comprises a coil, wherein a diameter of the coil of the NFC tag is in a range of 0.5-5.0 cm.

11. The pacifier according to claim 10, wherein a number of turns of the coil is at least two.

12. The pacifier according to claim 1, wherein the end portion comprises a planar outer surface, wherein a distance between the NFC tag and the planar outer surface is less than 3 mm.

13. A system comprising:
a pacifier according to claim 1;
a mobile device comprising an NFC device configured to read the radio wave signal containing the ID of the pacifier; and
a web-based service accessible via the Internet,
wherein the web-based service is configured to:
receive data from the mobile device, wherein the data contains the ID of the pacifier; and
send data related to the ID of the pacifier to the mobile device.

14. The system according to claim 13, wherein the mobile device has an installed program that allows a user of the mobile device to select from a list of predefined information, wherein the list includes information related to the pacifier selected from the group consisting of:
a production date of the pacifier;
chemical contents of materials that the pacifier is made from;
a location of a production site of the pacifier;
energy consumption relevant for production and/or transportation of the pacifier; and
carbon dioxide emissions relevant for the production and/or transportation of the pacifier.

15. A method for providing a unique ID of a pacifier having a base portion, a body portion, and a nipple attached to the body portion, the method comprising:
providing a pacifier that comprises a Near Field Communication (NFC) tag arranged in a such a position that when the NFC tag is triggered by an electromagnetic pulse from a NFC reader device integrated in a mobile device, the NFC tag generates a radio wave signal containing a unique ID of the pacifier back to the mobile device;
bringing the NFC device of the mobile device into a sufficiently small distance from the NFC tag so that the NFC tag generates a radio wave signal containing the unique ID of the pacifier; and
receiving the ID of the pacifier by the mobile device,
wherein the pacifier comprises an end portion attached to or integrated into the body portion, wherein the NFC tag is:
a) integrated in the body portion;
b) arranged at or attached to an end face of the body portion that is covered by the end portion; or
c) placed on, attached to, or integrated into the end portion.

16. The method according to claim 15, further comprising:
sending the unique ID of the pacifier to a web-based service that is configured to receive data from the mobile device, wherein the data contains the unique ID of the pacifier;
ensuring that the web-based service sends data related to the unique ID of the pacifier to the mobile device.

17. The method according to claim 16, further comprising:
running a program installed on the mobile device, wherein the installed program is configured to allow a user of the mobile device to select from a list of predefined information, wherein the list includes information related to the pacifier selected from the group consisting of:
a production date of the pacifier;
chemical contents of materials that the pacifier is made from;
a location of a production site of the pacifier;
energy consumption relevant for production and/or transportation of the pacifier; and/or
carbon dioxide emissions relevant for the production and/or transportation of the pacifier.

18. A method for recalling a plurality of pacifiers, the method comprising:
(i) providing one of the plurality of pacifiers according to claim 1;
(ii) bringing the NFC device of the mobile device into a sufficiently small distance from the NFC tag so that the NFC tag generates the radio wave signal containing the ID of the pacifier;
(iii) receiving the ID of the pacifier by the mobile device;
(iv) sending the ID of the pacifier to a web-based service that is configured to receive data from the mobile device, wherein the data contains the ID of the pacifier;
(v) receiving the ID at the web-based service;
repeating steps (i)-(v); and
sending recall information to each of the mobile devices via the web-based service.

19. A method for manufacturing the pacifier of claim 1, the method comprising:
arranging the NFC tag between the body portion and the end portion; and
joining the body portion and the end portion by a sealing procedure.

20. The method according to claim 19, wherein the sealing procedure is ultrasonic welding.
